(19) United States
(12) Patent Application Publication
(10) Pub. No.: US 2004/0082851 A1
(43) Pub. Date: Apr. 29, 2004

(54) Tunable electromagnetic device and method of use

(76) Inventors: Mehmet Bilgen, Houston, TX (US); Ponnada A. Narayana, Bellaire, TX (US)

Correspondence Address: Gary R. Maze, Duane Morris LLP, Suite 500, One Greenway Plaza, Houston, TX, 77046, US

(21) Appl. No.: 10/282,830

(22) Filed: Oct. 29, 2002

(51) Int. Cl. A61B 5/05

(52) U.S. Cl. 600/424; 600/407

(57) ABSTRACT

A device and method for tuning an element reactive in the presence of an external electromagnetic field as disclosed. The device comprises the reactive element, an electromagnetic field generator, and an external tuner disposed distally from the reactive element such that the electromagnetic field generator is intermediate the external tuner. A frequency at which the reactive element, inductively coupled to the electromagnetic field generator, will be responsive is tuned using the external tuner and adjusting a distance in a predetermined plane between the external tuner and the electromagnetic field generator. It is emphasized that this abstract is provided to comply with the rules requiring an abstract which will allow a searcher or other reader to quickly ascertain the subject matter of the technical disclosure. It is submitted with the understanding that it will not be used to interpret or limit the scope of meaning of the claims.

### FIELD OF THE INVENTION

This invention concerns tuning a reactive element in the presence of an electromagnetic field.

### BACKGROUND OF THE INVENTION

Magnetic resonance imaging (“MRI”) is a well known, highly useful technique for diagnosing abnormalities in biological tissue. In certain situations, implantable reactive devices such as MRI coils are used. These implanted MRI coils may be inductively coupled to an external coil to allow in vivo images of spatially localized biological tissue.

The quality of the magnetic resonance image is directly related to the characteristics of the receiving antenna. Significant electrical characteristics of the antenna include sensitivity, Q factor and the signal-to-noise ratio. Additionally, implanted coils may lack the necessary flexibility to tune their frequency of response when implanted.

Certain tunable, implantable MRI coils have been suggested in the prior art, but these typically involve complicated tuning circuitry, e.g. tuning circuitry based on capacitor-inductor combinations with or without diodes.

### SUMMARY

A device and method for tuning an element reactive in the presence of an external electromagnetic field, such as might be used in biological research or materials testing, are disclosed where the device comprises a reactive element such as an implantable coil, an electromagnetic field generator such as a radio frequency field generator, and an external tuner which may have a plate geometry. The electromagnetic field generator is disposed intermediate the external tuner and the reactive element.

A frequency at which the reactive element, inductively coupled to the electromagnetic field generator, will be responsive is tuned using the external tuner by adjusting a distance in a predetermined plane between the external tuner and the electromagnetic field generator.

It is noted that the scope of protection is not limited by the summary of an exemplary embodiment set out above, but is only limited by the claims.

### BRIEF DESCRIPTION OF THE DRAWINGS

These and other features, aspects, and advantages of the present invention will become more fully apparent from the following description, appended claims, and accompanying drawings in which:

FIG. 1 is a schematic overview of an embodiment of the present invention;

FIG. 2 is a flowchart of an exemplary tuning method; and

FIG. 3 is a flowchart of an exemplary tuning method in vivo.

### DETAILED DESCRIPTION OF THE PREFERRED EMBODIMENT

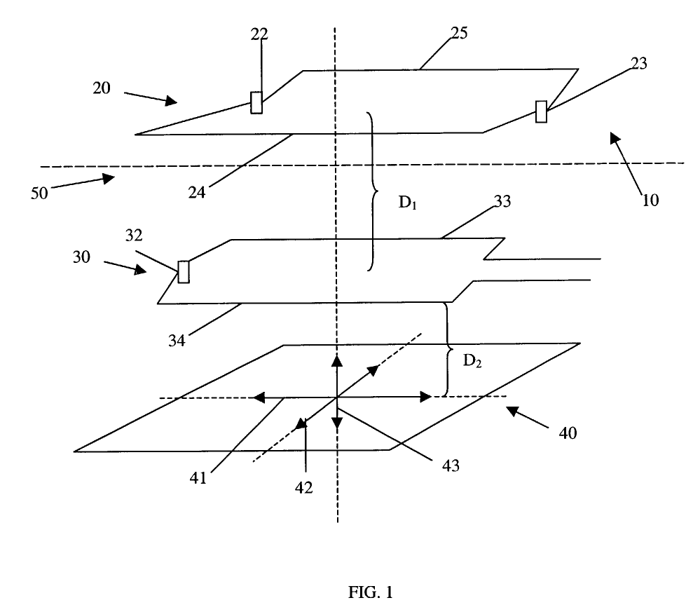

Referring now to FIG. 1, tunable electromagnetic device 10 comprises first element 20; electromagnetic field generator 30; and external tuner 40. In a preferred embodiment, tunable electromagnetic device 10 is reactive to an electromagnetic field at a frequency of the electromagnetic field.

In a preferred embodiment, first element 20 is a device that is responsive to an electromagnetic field at a desired frequency. In an exemplary embodiment, the frequency is one which is suitable for use with an magnetic resonance imaging (“MRI”) device (not shown in the figures) and first element 20 is a coil. Although first element 20 is typically reactive to the electromagnetic field at a single, predetermined frequency, it may also be reactive within a range of frequencies, to multiple frequencies, or to multiple simultaneous frequencies of the electromagnetic field.

For biological uses, first element 20 comprises a bio-compatible material adapted to be implanted within organic tissue. In a currently preferred embodiment, Silicone Elastomer MDX4-4210 available from Factor II, Incorporated, Lakeside, Ariz. is a preferred biocompatible material.

As will be understood by those of ordinary skill in the electronic arts, first element 20 may comprise one or more inductors 24,25 and one or more capacitors 22,23 which may be connected in series and/or in parallel to form an open circuit or a closed circuit. For embodiments comprising inductor 24,25 and capacitor 22,23, at least one inductor 24,25 may be connected in series to at least one capacitor 22,23. Additional passive and active electronic devices may be present, e.g. integrated circuits, diodes, and the like, or combinations thereof.

In a preferred embodiment, electromagnetic field generator 30 is inductively coupled to first element 20. Electromagnetic field generator 30 comprises at least one inductor 33,34 disposed intermediate first element 20 and external tuner 40. In a preferred embodiment, electromagnetic field generator 30 further comprises capacitor 32 connected in series to inductor 34,35. Electromagnetic field generator 30 may be disposed at a distance D₁ from first element 20 and a distance D₂ from external tuner 40.

External tuner 40 is disposed proximate first element 20 in a predetermined plane 41,42,43. For biological uses, first element 20 is disposed within an organic tissue and external tuner 40 is disposed outside the organic tissue.

In the preferred embodiment, external tuner 40 is a passive device that comprises a conductive metal, typically manufactured in a plate form having an approximately parallelogram shape, although other shapes and configurations may be used. The metal may comprise copper, a non-magnetic metal, or a combination thereof.

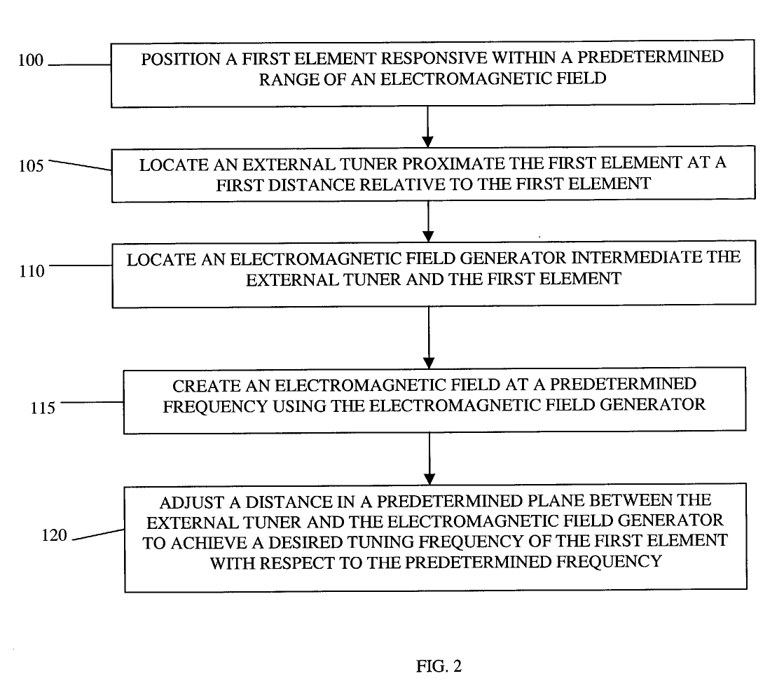

In the operation of a preferred embodiment, referring now to FIG. 2, tunable electromagnetic device 10 (FIG. 1) may be tuned to a desired frequency or range of frequencies at which first element 20 (FIG. 1), inductively coupled to electromagnetic field generator 30 (FIG. 1), will be responsive.

First element 20 (FIG. 1), which is initially responsive within a first predetermined range of an electromagnetic field, is positioned, step 100, at an initial position. For in vivo use, the initial position may be within a targeted biological tissue. External tuner 40 (FIG. 1) may be positioned to a location proximate first element 20 at a first distance D₁+D₂ (FIG. 1) relative to first element 20, at step 105.

Electromagnetic field generator 30 (FIG. 1) may be disposed intermediate external tuner 40 (FIG. 1) and first element 20 (FIG. 1), step 110, such that electromagnetic field generator 30 is positioned a distance D₁ (FIG. 1) from first element 20 and a distance D₂ (FIG. 1) from external tuner 40 (FIG. 1). An electromagnetic field may be generated at a predetermined frequency using electromagnetic field generator 30, step 115.

Distance D₂ (FIG. 1) may be adjusted, at step 120, in a predetermined plane such as defined by one or more axes 41,42,43 (FIG. 1) to achieve a desired tuning frequency of first element 20 (FIG. 1) with respect to a desired frequency. In a preferred embodiment, electromagnetic field generator 30 generates a radio frequency electromagnetic field suitable for use with an MRI device (not shown in the figures).

For example, distance D₂ (FIG. 1) may be adjusted to achieve a desired tuning frequency of first element 20 (FIG. 1) with respect to a single frequency, or with respect to within a predetermined range of frequencies, to multiple frequencies, or to multiple simultaneous frequencies of the electromagnetic field.

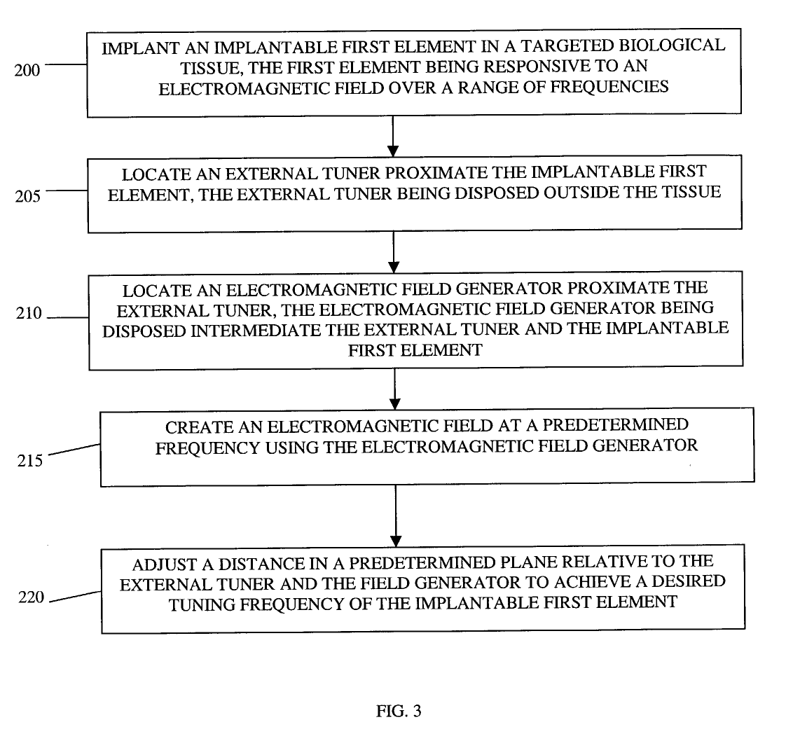

In a currently preferred embodiment, tunable electromagnetic device 10 (FIG. 1) may be used for biological uses such as in vivo research. For such an embodiment, referring now to FIG. 3, first element 20 (FIG. 1) is disposed in vivo and inductively coupled to external electromagnetic field generator 30 (FIG. 1), displaced a distance of D₁ (FIG. 1) from first element 20, for in vivo resolution of a spatially localized biological tissue.

In this embodiment, first element 20 (FIG. 1) is implantable in a targeted biological tissue, shown at dashed line 50 in FIG. 1. First element 20 is implanted, at step 200, in the targeted biological tissue. Over time, due to biological and/or chemical processes, first element 20 may change in its responsiveness to an electromagnetic field over a range of frequencies.

External tuner 40 (FIG. 1) may be located, at step 205, proximate to first element 20 (FIG. 1) where external tuner 40 (FIG. 1) is disposed outside the targeted biological tissue, e.g. outside a host organism or host body. Electromagnetic field generator 30 (FIG. 1) is located, at step 210, at a distance D₂ (FIG. 1) from external tuner 40 and external to spatially localized biological tissue, generally shown at 50 in FIG. 1, at a distance D₁ (FIG. 1) from first element 20. An electromagnetic field may be created, at step 215, at a desired, predetermined frequency using electromagnetic field generator 30. Distance D₂ (FIG. 1) may be adjusted in one or more predetermined planes relative to external tuner 40, such as defined by axes 41,42,43 (FIG. 1), and electromagnetic field generator 30 to achieve a desired tuning frequency of implanted first element 20, step 220.

A frequency response of first element 20 (FIG. 1) may be monitored in the presence of a radio frequency electromagnetic field while adjusting distance D₂ (FIG. 1) during the monitoring to achieve the desired tuning frequency of first element 20. Additionally, monitoring and adjusting may occur at a predefined time interval during use or on an as needed basis.

It will be understood that various changes in the details, materials, and arrangements of the parts which have been described and illustrated above in order to explain the nature of this invention may be made by those skilled in the art without departing from the principle and scope of the invention as recited in the following claims.

What is claimed is:

1) A tunable electromagnetic device, comprising:
a. a first element, reactive to an electromagnetic field at a frequency of the electromagnetic field;
b. an external tuner disposed proximate the first element in a predetermined plane; and
c. an electromagnetic field generator, the field generator comprising an inductor disposed intermediate the first element and the external tuner, the electromagnetic field generator inductively coupled to the first element.

2) The tunable electromagnetic device of claim 1, wherein:
a. the first element is reactive to the electromagnetic field at at least one of (i) a single frequency, (ii) a range of frequencies, (iii) multiple frequencies, or (iv) multiple simultaneous frequencies of the electromagnetic field.

3) The tunable electromagnetic device of claim 1, wherein:
a. the first element comprises a bio-compatible material adapted to be implanted within organic tissue.

4) The tunable electromagnetic device of claim 1, wherein:
a. the first element is disposed within an organic tissue; and
b. the external tuner is disposed outside the organic tissue.

5) The tunable electromagnetic device of claim 1, wherein:
a. the first element comprises an inductor and a capacitor connected in series to form at least one of (i) an open circuit and (ii) a closed circuit.

6) The tunable electromagnetic device of claim 1, wherein:
a. the first element comprises a plurality of inductors and a plurality of capacitors, at least one inductor connected in series to at least one capacitor.

7) The tunable electromagnetic device of claim 1, wherein:
a. the first element is responsive to an electromagnetic field at a frequency suitable for use with a magnetic resonance imaging “MRI” device.

8) The tunable electromagnetic device of claim 1, wherein:
a. the electromagnetic field generator further comprises a capacitor connected in series to the inductor.

9) The tunable electromagnetic device of claim 1, wherein:
a. the external tuner is passive.

10) The tunable electromagnetic device of claim 1, wherein:
a. the external tuner is a conductive plate comprising a metal.

11) The tunable electromagnetic device of claim 9, wherein:
a. the metal is at least one of (i) copper or (ii) a non-magnetic metal.

12) A method of tuning a frequency at which a first element inductively coupled to a field generator will be responsive, comprising:
a. positioning a first element responsive within a predetermined range of an electromagnetic field;
b. locating an external tuner proximate the first element at a first distance relative to the first element;
c. disposing an electromagnetic field generator intermediate the external tuner and the first element;
d. creating an electromagnetic field at a predetermined frequency using the electromagnetic field generator; and
e. adjusting a distance in a predetermined plane between the external tuner and the electromagnetic field generator to achieve a desired tuning frequency of the first element with respect to the predetermined frequency.

13) The method of tuning a frequency of claim 12, wherein:
a. the distance is adjusted to achieve a desired tuning frequency of the first element with respect to at least one of (i) a single frequency, (ii) within a predetermined range of frequencies, (iii) multiple frequencies, or (iv) multiple simultaneous frequencies of the electromagnetic field.

14) The method of tuning a frequency of claim 12, wherein:
a. the electromagnetic field generator generates a radio frequency electromagnetic field suitable for use with an MRI device.

15) The method of claim 16, further comprising:
a. monitoring a frequency response of the first element in the presence of the electromagnetic field;
b. wherein the monitoring and adjusting occur at a predefined time interval during use.

16) A method of using a first element responsive to the presence of an electromagnetic field, the first element disposed in vivo and inductively coupled to an external electromagnetic field generator, for in vivo resolution of a spatially localized biological tissue, comprising:
a. implanting an implantable first element in a targeted biological tissue, the first element being responsive to an electromagnetic field over a range of frequencies;
b. locating an external tuner proximate the implantable first element, the external tuner being disposed outside the tissue;
c. locating an electromagnetic field generator proximate the external tuner, the electromagnetic field generator being disposed intermediate the external, tuner and the implantable first element;
d. creating an electromagnetic field at a predetermined frequency using the electromagnetic field generator; and
e. adjusting a distance in a predetermined plane relative to the external tuner and the field generator to achieve a desired tuning frequency of the implantable first element.

17) The method of claim 16, further comprising:
a. monitoring a frequency response of the first element in the presence of a radio frequency electromagnetic field while adjusting the external tuner; and
b. adjusting the distance in the predetermined plane between the electromagnetic field generator and the external tuner achieve a desired tuning frequency of the implantable first element.

18) The method of claim 17, wherein:
a. the monitoring and adjusting occur at a predefined time interval during use.
